(19) United States
(12) Patent Application Publication
(10) Pub. No.: US 2021/0247409 A1
(43) Pub. Date: Aug. 12, 2021

(54) GRAPHENE-BASED SENSOR FOR DETECTING HEMOGLOBIN IN A BIOLOGICAL SAMPLE

(71) Applicant: Graphene-DX, Inc., Boston, MA (US)

(72) Inventors: Namal NAWANA, Weston, MA (US); Mehdi Abedi, Brighton, MA (US); Reza Mollaaghababa, Natick, MA (US)

(21) Appl. No.: 17/170,439

(22) Filed: Feb. 8, 2021

Related U.S. Application Data
(60) Provisional application No. 62/970,919, filed on Feb. 6, 2020.

(51) Int. Cl. G01N 33/72; G01N 33/574; B01L 3/00

(52) U.S. Cl. CPC G01N 33/726; G01N 33/57419; B01L 3/502715; B01L 3/50825; B01L 2300/0636; B01L 2200/027; B01L 2200/10; B01L 2200/16; B01L 2300/044; G01N 2333/805

(57) ABSTRACT

In one aspect, a sensor for detecting hemoglobin protein (e.g., human hemoglobin protein) in a sample is disclosed, which includes a graphene layer, a plurality of binding agents coupled to said graphene layer to generate a functionalized graphene layer, where the binding agents exhibit specific binding to a hemoglobin protein (e.g., to human hemoglobin protein) and a plurality of electrical conductors electrically coupled to said functionalized graphene layer for measuring an electrical property (e.g., DC electrical resistance) of said functionalized graphene layer. While in some embodiments such binding agents are monoclonal antibodies, in other embodiments they can be polyclonal antibodies.

### CROSS-REFERENCE TO RELATED APPLICATIONS

This application claims priority to U.S. Provisional Application No. 62/970,919 filed on Feb. 6, 2020, which application is herein incorporated by reference in its entirety.

### BACKGROUND

The present disclosure relates generally to a sensor and methods of using the sensor for detecting hemoglobin protein in a sample, and more particularly for detecting hemoglobin protein in a biological sample, such as human feces.

Colorectal cancer (CRC) accounts for 10.0% and 9.2% of all cancers in men and women, respectively. The survival rate of CRC varies significantly based on the stage of diagnosis. For example, five-year survival rate ranges from 90% for CRC detected at the localized stage; 70% for regional; and down to 10% in people with distant metastasis. Thus, diagnostic methods for early detection of CRC are highly in demand.

Colonoscopy remains the gold standard for CRC diagnosis. Fecal occult blood test (FOBT) is also employed as means of detecting hemoglobin in a fecal sample as a valuable screening tool.

There is still a need for improved sensors for detecting hemoglobin protein in a fecal sample, and particularly for low-cost, point-of-care sensors that can rapidly detect hemoglobin protein in a fecal sample.

### SUMMARY

In one aspect, a sensor for detecting hemoglobin in a sample is disclosed, which comprises a graphene layer, a plurality of anti-hemoglobin antibodies and/or aptamers coupled to the graphene layer to generate a functionalized graphene layer, and a plurality of electrical conductors electrically coupled to said functionalized graphene layer for measuring at least one electrical property of said functionalized graphene layer.

The anti-hemoglobin antibodies and/or aptamers comprise antibodies and/or aptamers exhibiting specific binding to hemoglobin protein. In some embodiments, the antibodies include anti-human hemoglobin antibodies. In some embodiments, the antibodies are coupled to the underlying graphene layer using a plurality of linkers. In some such embodiments, each linker is attached at one end thereof to the graphene layer, e.g., via a π-π bond, and at another end to at least one hemoglobin protein, e.g., via a covalent bond. By way of example, in some embodiments, the linker can be 1-pyrenebutonic acid succinimidyl ester.

In some embodiments, the sensor can further include a reference electrode for applying a reference AC voltage and/or current (herein also referred to as an “AC signal”), and in some embodiments as well as a DC offset voltage (e.g., a DC ramp voltage) to the functionalized graphene layer during data acquisition. By way of example, the reference AC signal can have a frequency in a range of about 1 kHz to about 1 MHz, such as in a range of about 10 kHz to about 100 kHz, or in a range of about 50 kHz to about 200 kHz, or in a range of about 200 kHz to about 300 kHz, or in a range of about 400 kHz to about 700 kHz, and the amplitude of the applied AC voltage (e.g., the peak-to-peak amplitude) can be, for example, in a range of about 100 millivolts to about 3 volts, e.g., in a range of about 1 volt to about 2 volts.

As noted above, in some embodiments, a DC ramp voltage is applied to the AC electrode, together with the AC voltage, during data acquisition. The DC ramp voltage can extend, for example, from about −10 volts to about +10 volts, e.g., in a range of about −5 volts to about +5 volts, or in a range of about −3 volts to about +3 volts, or in a range of about −1 volt to about +1 volt.

While in some embodiments the reference electrode can be positioned above the functionalized graphene layer, in other embodiments the reference electrode can be positioned on the substrate on which the graphene layer is disposed. In some such embodiments, the reference electrode can at least partially surround the functionalized graphene layer.

In many embodiments, the sample includes a biological sample, such as feces (e.g., human feces), urine or blood.

In some embodiments, the sensor is configured to have a detection sensitivity (limit-of-detection (LOD)) of at least 10 μg·Hb/g·feces for detecting hemoglobin protein in human feces.

In some embodiments, a sensor according to the present teachings can include at least one microfluidic channel for guiding a sample from an inlet port to an outlet port that is in communication with a graphene-based sensing element according to the present teachings. In some embodiments, the microfluidic channel can have passive and/or active mixing elements for mixing the sample.

In a related aspect, a method of detecting hemoglobin in a biological sample is disclosed, which includes applying the sample to a graphene layer that is functionalized with a plurality of anti-hemoglobin antibodies (e.g., anti-human hemoglobin antibodies) and/or aptamers, measuring at least one electrical property of the functionalized graphene layer, and using the measured electrical property to determine whether hemoglobin is present in the sample. In some embodiments, the measured electrical property of the graphene layer can be, for example, electron mobility, electrical impedance (e.g., DC or AC electrical resistance or both), and electrical capacitance.

In some embodiments, the biological sample can be, for example, human feces, urine or blood.

In a related aspect, a method of fabricating a sensor for detecting hemoglobin in a biological sample is disclosed, which comprises attaching a plurality of linkers to a graphene layer deposited on an underlying substrate, and covalently coupling a plurality of antibodies and/or aptamers exhibiting specific binding to hemoglobin protein (e.g., anti-human hemoglobin protein) to the linkers.

In a related aspect, a disposable cartridge for detecting hemoglobin in a biological sample (e.g. human feces) is disclosed, which comprises a microfluidic component having an inlet port for receiving a sample and an exit port. A sensor is fluidically coupled to the microfluidic component to receive at least a portion of the sample from the exit port. The sensor can include a graphene layer, and a plurality of anti-hemoglobin antibodies and/or aptamers that are coupled to the graphene layer to generate an antibody and/or aptamer-functionalized graphene layer. A plurality of electrical conductors are electrically coupled to the functionalized graphene layer for measuring an electrical property of the functionalized graphene layer in response to exposure of the antibody and/or aptamer-functionalized graphene layer to a sample.

In some embodiments, the microfluidic component is formed of a polymeric material, such as PDMS or PMMA.

In some embodiments, a sensor according to the present teachings can be employed to detect hemoglobin in human feces and the results can be employed, by itself, or in combination with genetic testing, for detection of colorectal cancer and/or pre-malignant colorectal neoplasia.

In a related aspect, a method of screening for any of colorectal cancer and pre-malignant colorectal neoplasia is disclosed, which comprises collecting a stool sample from a subject, dissolving the collected stool sample in one or more reagents (e.g., a buffer solution) to form a testing sample, introducing the testing sample onto a sensing element comprising a graphene layer functionalized with a plurality of antibodies and/or aptamers exhibiting specific binding to hemoglobin protein, measuring at least one electrical property of said antibody and/or aptamer-functionalized graphene layer in response to exposure thereof to said testing sample, and determining whether hemoglobin protein above a threshold corresponding to the limit-of-detection (LOD) of the sensing element is present in said testing sample. In some embodiments, in addition to dissolving the stool sample in one or more reagents, the sample can also be subjected to mixing.

Optionally, the method can further comprise quantifying the concentration of the hemoglobin protein in the testing sample. The quantified concentration of the hemoglobin protein can then be correlated with the presence or absence of colorectal cancer or pre-malignant colorectal neoplasia.

In some embodiments, one or more reagents employed to form the testing sample is free (or substantially free) of a stabilizing reagent.

In some embodiments, at least one genetic screening method is utilized in combination with the detection of the hemoglobin protein to determine the presence or absence of colorectal cancer or pre-malignant colorectal neoplasia. In other embodiments, only hemoglobin protein is employed as a biomarker for the detection of colorectal cancer or pre-malignant colorectal neoplasia.

In some embodiments, a sensor according to the present teachings can include at least two sensing units, where one of the sensing units is configured for the detection of hemoglobin within a biological sample (e.g., feces), and another sensing unit is configured to detect one or more genetic biomarkers associated with colorectal cancer and/or pre-malignant colorectal dysplasia. In some such embodiments, the hemoglobin-detecting sensing unit can have a structure such as those disclosed herein and the biomarker sensing unit can have a graphene layer that is functionalized with a catalytically inactive CRISPR complex with a sgRNA having a oligonucleotide sequence that is complementary with a DNA sequence of interest. If the DNA sequence (e.g., a DNA sequence having a target mutation, e.g., a mutation associated with colorectal cancer in the KRAS gene) is present in a sample (e.g., a fecal sample), the binding of that DNA sequence to the sgRNA can cause a change in at least one electrical property of the functionalized graphene layer, which can be measured and analyzed as discussed below to identify the presence of the mutation in the genome. In some such embodiments, the identification of hemoglobin above a selected threshold together with one or more target mutations and/or specific sequences can signal the presence of colorectal cancer or pre-malignant colorectal dyplasia.

A variety of known genetic screening methods, including those disclosed herein, can be employed in the practice of the present teachings. As discussed in more detail below, some such genetic screening methods can rely on the detection of KRAS mutations and/or aberrant methylation.

Further understanding of various aspects of the present teachings can be obtained with reference to the following detailed description in conjunction with the associated drawings, which are described briefly below.

### BRIEF DESCRIPTION OF THE DRAWINGS

FIG. 1A schematically depicts a disposable cartridge according to an embodiment for detecting hemoglobin in a sample,

FIG. 1B schematically depicts a graphene-based sensor employed in the cartridge depicted in FIG. 1A,

FIG. 2 is a schematic view of a graphene-based sensor according to an embodiment including a plurality of metallic pads for measuring an electrical property thereof in response to interaction with a sample under study,

FIG. 3A depicts a circuit diagram of an example of a voltage-measuring device that can be employed for measuring a voltage induced across an antibody-functionalized graphene layer in response to application of a current thereto,

FIG. 3B schematically depicts an analyzer in communication with the voltage-measuring device shown in FIG. 3A for receiving the voltage measured by the voltage-measuring device as well as the current applied to the antibody-functionalized graphene layer,

FIG. 3C depicts an example of implementation of the analyzer shown in FIG. 3A,

FIG. 4A schematically depicts a sensor according to an embodiment, which includes an AC reference electrode,

FIG. 4B schematically depicts a sensor according to an embodiment, which includes an AC reference electrode on the substrate,

FIG. 4C schematically depicts a combination of a ramp voltage and an AC voltage applied to the reference electrode of a sensor according to an embodiment of the present teachings,

FIG. 5 schematically depicts an array of graphene-based sensor in accordance with an embodiment,

FIG. 6A schematically depicts a hydroxyl-functionalized graphene layer,

FIG. 6B schematically depicts a hydroxyl-functionalized graphene layer to which antibodies are attached,

FIG. 7A schematically depicts a serpentine microfluidic channel that can be employed in some embodiments of a sensor according to the present teachings to cause passive mixing of a sample passing therethrough,

FIG. 7B schematically depicts a spiral microfluidic channel that can be employed in some embodiments of a sensor according to the present teachings to cause passive mixing of a sample passing therethrough,

FIG. 7C schematically depicts a sensor according to an embodiment in which a microfluidic channel in which active mixing elements are incorporated guides a sample from an inlet port to a graphene-based sensing element according to the present teachings,

FIGS. 8A, 8B, and 8C schematically depict a system according to an embodiment of the present teachings for collecting a stool sample and testing the sample for presence of hemoglobin protein therein,

FIG. 9 schematically depicts that in some embodiments the lid of the system shown in FIGS. 8A, 8B, and 8C can include a communications module for communicating via a variety of wireless protocols to local and remote devices,

FIGS. 10A, 10B, 10C, and 10D schematically depict a sample collection and processing system according to an embodiment of the present teachings,

FIGS. 11 and 12 schematically show an embodiment of the graphene-based sensor according to the present teachings.

FIG. 13 schematically depicts an implementation of the measurement scheme for the embodiments described in FIGS. 11 and 12.

FIG. 14 schematically depicts another implementation of the measurement scheme for the embodiment described in FIGS. 11 and 12.

FIG. 15 schematically shows another embodiment of the sensing unit of the graphene-based sensor according to the present teachings.

FIG. 16 shows experimental results comparing electron mobilities of graphene layers functionalized with hemoglobin antibodies and isotype control antibodies when exposed to hemoglobin-containing sample.

### DETAILED DESCRIPTION

The present disclosure relates generally to a graphene-based sensor that can be employed for detecting a hemoglobin protein in a sample, such as human feces. Various terms are used herein in accordance with their ordinary meanings in the art. The term “about” as used herein denotes a variation of at most 5%, 10%, 15%, or 20% around a numerical value. The term “limit of detection,” as used herein refers to a minimum concentration of an analyte, e.g., hemoglobin protein in this embodiment, that can be positively detected using a sensor according to the present teachings.

In one aspect, the present disclosure provides teachings that allow the detection of hemoglobin in a sample under investigation via the binding of hemoglobin to a binding agent that is coupled to a graphene layer to generate a functionalized graphene layer and measuring a change in at least one electrical property of the functionalized graphene layer. Some example of such binding agents include, without limitation, an aptamer, an antibody, an antibody fragment, etc. In the following description, for ease of explanation, the term “antibody” is intended to refer to any suitable binding agent, i.e., any binding agent that exhibits specific binding to hemoglobin.

The term “antibody,” as used herein, may refer to a polypeptide that exhibit specific binding affinity, e.g., an immunoglobulin chain or fragment thereof, comprising at least one functional immunoglobulin variable domain sequence. An antibody encompasses full length antibodies and antibody fragments. In some embodiments, an antibody comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody.

The term “antibody” also encompasses whole or antigen binding fragments of domain, or signal domain, antibodies, which can also be referred to as “sdAb” or “VHH.” Domain antibodies comprise either VH or VL that can act as stand-alone, antibody fragments. Additionally, domain antibodies include heavy-chain-only antibodies (HCAbs). Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

The term “aptamer,” as used herein, refers to an oligonucleotide or a peptide molecule that exhibits specific binding to a target molecule. Aptamers are typically created by selecting them from a large random pool of oligonucleotide or peptide sequences, but natural aptamer do also exist.

The term “oligonucleotide binding element” as used herein refers to any of a protein, a peptide and/or an oligonucleotide that exhibits specific binding to a target oligonucleotide, such as an RNA or a single strand DNA segment.

The term “electrical property” as used herein may include electron mobility, electrical impedance (e.g., DC or AC electrical resistance or both), and/or electrical capacitance.

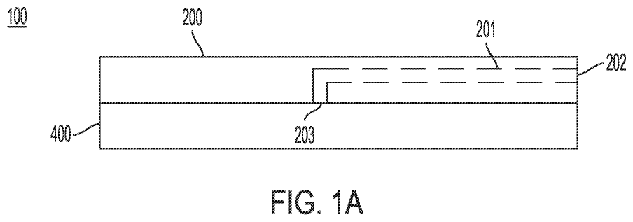

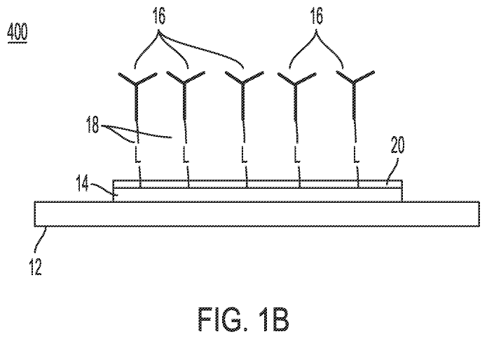

FIG. 1A schematically depicts a cartridge 100 (herein also referred to as a cassette) according to an embodiment that can be employed to detect hemoglobin protein in a sample, e.g., human feces. In many embodiments, the cartridge 100 is a single-use and disposable cartridge.

The cartridge 100 includes a microfluidic delivery component 200 for delivering a sample under investigation to a sensor 400. In this embodiment, the microfluidic delivery component 200 includes at least one fluidic channel 201 that extends from an inlet port 202 through which a sample can be introduced into the microfluidic component to an outlet port 203 via which the sample can be delivered to the sensor 400. In some embodiments, the microfluidic channel can function based on capillary action. In some embodiments, the microfluidic delivery component 200 can be formed of a polymeric material, such as PDMS (polydimethylsiloxane) or PMMA (polymethyl methacrylate), and the microfluidic channel can be formed via etching or other known techniques in the art.

As shown schematically in FIG. 1B, in this embodiment, the sensor 400 includes a graphene layer 14 that is disposed on an underlying substrate 12. While in some embodiments the substrate can be a semiconductor in other embodiments, it can be a polymeric substrate. By way of example, in some embodiments, the substrate can be a silicon substrate while in other embodiments it can be a plastic substrate. For example, the underlying substrate can be formed of PDMS. Yet, in other embodiments, the underlying substrate can be a metallic substrate, such as a copper substrate. In some embodiments, an SiO₂ layer separates the graphene layer from an underlying silicon substrate.

In this embodiment, the graphene layer is functionalized with a plurality of binding agents (e.g., antibodies and/or aptamers) 16 that exhibit specific binding to hemoglobin protein, e.g., human hemoglobin protein. Hemoglobin is composed of four protein chains, two alpha chains and two beta chains, each with a ring-like heme group containing an iron atom. Oxygen binds reversibly to these iron atoms and is transported through blood. By way of example, an anti-human hemoglobin antibody can be obtained from Sigma Aldrich under the product code H4890-2ML.

The binding agents 16 can be monoclonal or polyclonal antibodies, as discussed in more detail below. As shown schematically in FIG. 1B, a variety of linker molecules 18 can be employed for coupling the anti-hemoglobin antibodies to the underlying graphene layer. By way of example, in some embodiments, 1-pyrenebutonic acid succinimidyl ester is employed as a linker to facilitate the coupling of the anti-hemoglobin antibodies to the underlying graphene layer. In this embodiment, the plurality of anti-hemoglobin antibodies can cover a fraction of, or the entire, surface of the graphene layer. In various embodiments, the fraction can be at least about 60%, at least about 70%, at least about 80%, or 100% of the surface of the graphene layer. The remainder of the surface of the graphene layer (i.e., the surface areas not functionalized with the anti-hemoglobin antibodies) can be passivated via a passivation layer 20. By way of example, the passivation layer can be formed by using Tween 20, BLOTTO, BSA (Bovine Serum Albumin), gelatin or 3 mM APA (amino-PEGS-alcohol), though other reagents may also be used. The passivation layer can inhibit, and preferably prevent, the interaction of a sample of interest introduced onto the graphene layer with areas of the graphene layer that are not functionalized with the anti-hemoglobin antibodies. This can in turn lower the noise in the electrical signals that will be generated as a result of the interaction of the analyte of interest with the antibody molecules.

By way of example, in some embodiments, a graphene layer formed on an underlying substrate (e.g., plastic, a semiconductor, such as silicon, or a metal substrate, such as a copper film) can be incubated with the linker molecule (e.g., a 5 mM solution of 1-pyrenebutonic acid succinimidyl ester) for a few hours (e.g., 2 hours) at room temperature.

The linker modified graphene layer can then be incubated with the antibody of interest in a buffer solution (e.g., NaCO₃—NaHCO₃ buffer solution (pH 9)) at a selected temperature and for a selected duration (e.g., 7-10 hours at 4° C.), followed by rinsing with deionized (DI) water and phosphate buffered solution (PBS). In order to quench the unreacted succinimidyl ester groups, the modified graphene layer can be incubated with ethanolamine (e.g., 0.1 M solution at a pH of 9 for 1 hour).

Subsequently, the non-functionalized graphene areas can be passivated via a passivation layer, such as the passivation layer 20 schematically depicted in FIG. 1B. By way of example, the passivation of the non-functionalized portions of the graphene layer can be achieved, e.g., via incubation with 0.1% Tween 20.

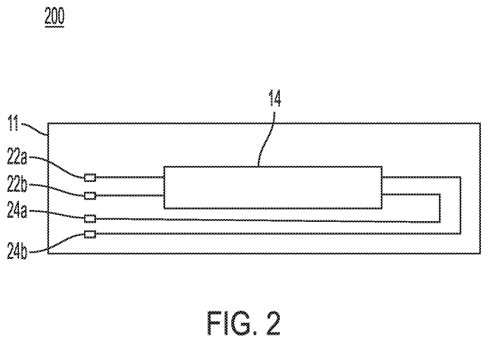

FIG. 2 shows a sensor 200 according to some embodiments, which includes electrically conductive pads 22a, 22b, 24a and 24b, that allow four point measurement of modulation of an electrical property of the functionalized graphene layer 14 in response to interaction of hemoglobin protein present in a sample with the anti-hemoglobin antibodies coupled to the graphene layer 14. In particular, in this embodiment, the conductive pads 22a/22b are disposed on the substrate 11 and electrically coupled to one end of the functionalized graphene layer 14 and the conductive pads 24a/24b are disposed on the substrate 11 and electrically coupled to the opposed end of the functionalized graphene layer 14 to allow measuring a change in an electrical property of the underlying graphene layer 14 caused by the interaction of hemoglobin protein in a sample under study with the anti-hemoglobin antibodies that are coupled to the graphene layer 14. By way of example, in this embodiment, a change in the DC resistance of the underlying graphene layer 14 can be monitored to determine the presence of hemoglobin protein in a sample under study. In other embodiments, a change in electrical impedance of the graphene layer 14 characterized by a combination of DC resistance and capacitance of the graphene/antibody system can be monitored to determine whether hemoglobin protein is present in a sample under study. The electrically conductive pads can be formed using a variety of metals, such as copper and copper alloys, among others.

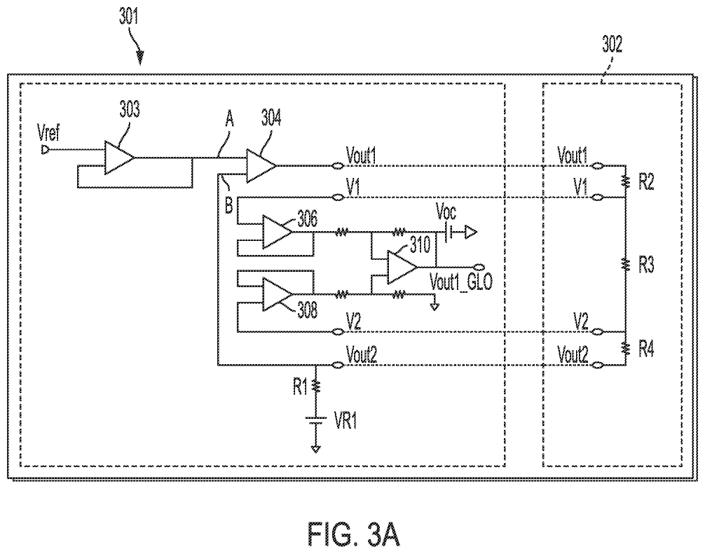

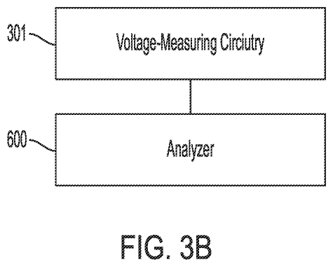

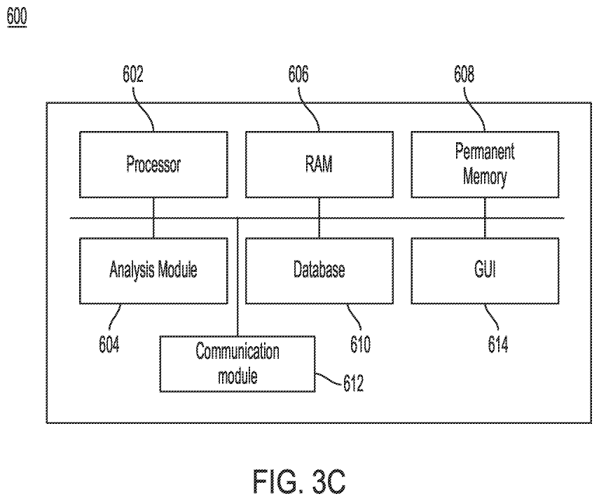

By way of example, FIG. 3A schematically depicts a voltage measuring circuitry 301 that can be employed in some embodiments of the present teachings. This figure shows a sensor 302 as an equivalent circuit corresponding to an antibody-functionalized graphene layer. A fixed voltage V (e.g., 1.2 V) is generated at the output of a buffer operational amplifier 303. This voltage is applied to one input (A) of a downstream operational amplifier 304 whose other input B is coupled to VR1 ground via a resistor R1. The output of the operational amplifier 304 (Vₒᵤₜ₁) is coupled to one end of the sensor 302 and the end of the resistor R1 that is not connected to VR1 ground is coupled to the other end of the sensor 302 (in this schematic diagram, resistor R2 denotes the resistance between two electrode pads at one end of the equivalent sensor 302, resistor R3 denotes the resistance of the graphene layer extending between two inner electrodes of the sensor, and resistor R4 denotes the resistance between two electrode pads at the other end of the sensor). As the operational amplifier maintains the voltage at the end of the resistor R1 that is not connected to VR1 ground at the fixed voltage applied to its input (A), e.g., 1.2 V, a constant current source is generated that provides a constant current flow through the sensor 302 and returns to ground via the resistor R1 and VR1.

The voltage generated across the antibody-functionalized graphene layer is measured via the two inner electrodes of the sensor. Specifically, one pair of the inner electrode pads is coupled to a buffer operational amplifier 306 and the other pair is coupled to the other buffer operational amplifier 308. The outputs of the buffer operational amplifiers are applied to the input ports of a differential amplifier 310 whose output port provides the voltage difference across the antibody-functionalized graphene layer. This voltage difference (Vₒᵤₜ₁−GLO) can then be used to measure the resistance exhibited by the antibody-functionalized graphene layer. The current forced through R3 is set by I=(Vref−VR1)/R1, where the value of VR1 is digitally controlled. For each value of current I, the corresponding voltage (V_{out1_}GLO) is measured and stored. The resistance of the antibody-functionalized graphene layer can be calculated as the derivative of the voltage, V_{out1_}GLO, with respect to current I, i.e., R=dV/dI.

As shown schematically in FIG. 3B, in some embodiments, an analyzer 600 can be in communication with the voltage measuring circuitry 301 to receive the applied current and the measured voltage value and use these values to calculate the resistance of the antibody-functionalized graphene layer. The analyzer 600 can then employ the calculated resistance, e.g., a change in the resistance in response to exposure of the antibody-functionalized graphene layer to a sample under investigation, to determine, in accordance with the present teachings, whether the sample contains hemoglobin protein.

By way of example, as shown schematically in FIG. 3C, in this embodiment, the analyzer 600 can include a processor 602, an analysis module 604, a random access memory (RAM) 606, a permanent memory 608, a database 610, a communication module 612, and a graphical user interface (GUI) 614. The analyzer 600 can employ the communication module 612 to communicate with the voltage measuring circuitry 301 to receive the values of the applied current and the measured voltage. The communication module 612 can be a wired or a wireless communication module. The analyzer 600 further includes a graphical user interface (GUI) 614 that allows a user to interact with the analyzer 600.

The analysis module 604 can employ the values of a current applied to the antibody-functionalized graphene layer as well as the voltage induced across the graphene layer to calculate a change in the resistance of the antibody-functionalized graphene layer in response to exposure thereof to a sample under investigation (e.g., using Ohm's law). The instructions for such calculation can be stored in the permanent memory 608 and can be transferred at runtime to RAM 606 via processor 602 for use by the analysis module 604. The GUI 614 can allow a user to interact with the analyzer 600.

In some embodiments, the analyzer 600 can include an AC (alternating current) source of current, which can apply an AC current having a known amplitude and frequency to the graphene layer. In particular, various embodiments can advantageously use an AC voltage having a frequency in a range of about 1 kHz to about 1 MHz, e.g., in a range of about 10 kHz to about 500 kHz, or in a range of about 20 kHz to about 400 kHz, or in a range of about 30 kHz to about 300 kHz, or in a range of about 40 kHz to about 200 kHz. By way of example, the amplitude of the AC voltage applied to the reference electrode can be in a range of about 1 millivolt to about 3 volts, e.g., in a range of about 100 millivolts to about 2 volts, or in range of about 200 millivolts to about 1 volt, or in range of about 300 millivolts to about 1 volt, e.g., in a range of about 0.5 volts to 1 volt.

The analyzer 600 can further include an AC voltmeter circuitry for measuring the AC voltage induced across the graphene layer in response to the application of the AC current to the layer. By measuring the amplitude and/or phase shift of the induced AC voltage, the electrical impedance of the graphene layer can be determined in a manner known in the art. In other embodiments, an AC voltage having a fixed frequency and amplitude can be applied to the functionalized graphene layer and the current can be monitored for detecting specific binding of hemoglobin to the antibodies coupled to the graphene layer.

Further details regarding a suitable analyzer that can be employed in the practice of some embodiments of the present teachings can be found, e.g., in U.S. Pat. No. 9,664,674 titled “Device and Method for Chemical Analysis,” which is herein incorporated by reference in its entirety.

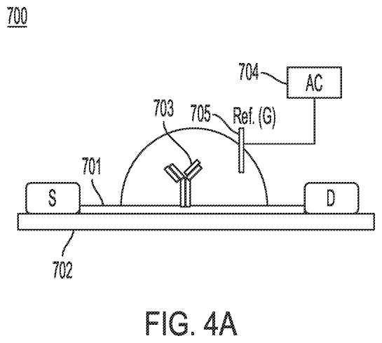

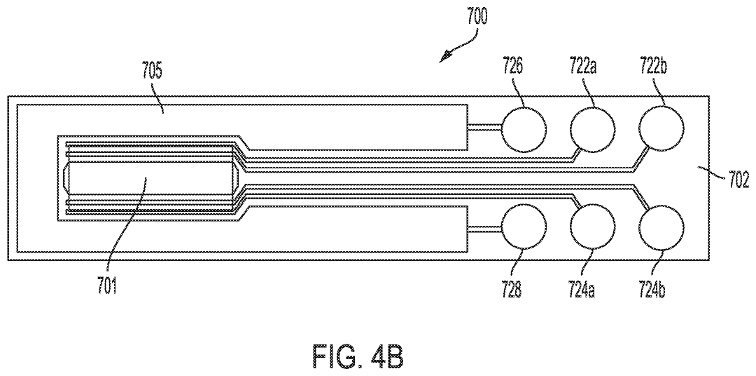

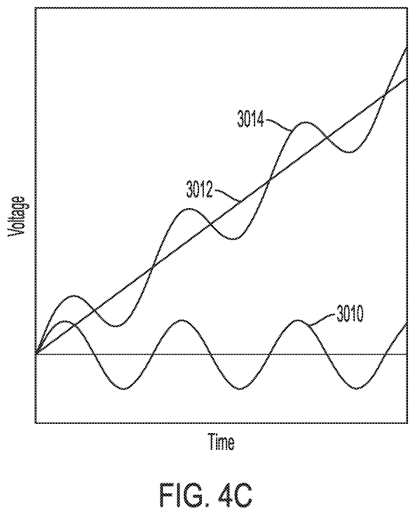

FIGS. 4A and 4B depict another embodiment of a sensor 700 according to the present teachings. Sensor 700 can include a graphene layer 701 that is disposed on an underlying substrate 702, e.g., a semiconductor substrate, and is functionalized with an antibody of interest 703, which can exhibit specific binding to a hemoglobin (such as human hemoglobin). A source electrode (S) and a drain electrode (D) are electrically coupled to the graphene layer to allow measuring a change in one or more electrical parameters of the functionalized graphene layer in response to interaction of the functionalized graphene layer with a sample. Referring to FIG. 4B, for four point measurement of modulation of an electrical property of the functionalized graphene layer 701, the sensor 700 can include electrically conductive pads 722a, 722b, 724a and 724b, similar to the embodiment shown in FIG. 2. The sensor 700 further includes a reference electrode (G) 705 that is disposed on the same substrate 702 as that on which the graphene layer 701 is disposed (in other words, the reference electrode 705 is in substantially same plane as the graphene layer 701). In some embodiments, the reference electrode 705 can substantially surround the graphene layer 701. The reference electrode 705 can be electrically connected to additional conductive pads 726 and 728 to allow application of an AC voltage as well as a DC ramp voltage to the reference electrode 705, e.g., in a manner discussed above.

In use, in some embodiments, a change in the electrical resistance of the functionalized graphene layer can be measured in response to the interaction of the functionalized graphene layer with a sample, e.g., human feces, to determine whether hemoglobin antibody is present in the sample. For example, when the sample contains hemoglobin protein above a certain concentration threshold, e.g., 10 μg·Hb/g·feces, the interaction of the hemoglobin protein with the antibodies coupled to the graphene layer can cause modulation of an electrical property of the graphene layer (e.g., DC resistance) and hence provide a signal indicative of the presence of hemoglobin protein in the sample.

In some embodiments, the application of an AC (alternating current) reference voltage via an AC voltage source 704 to the graphene layer can facilitate the detection of one or more electrical properties of the functionalized graphene, e.g., a change in its resistance in response to the interaction of the antibody with an analyte exhibiting specific binding to the antibody. In particular, in some embodiments, the application of an AC voltage having a frequency in a range of about 1 kHz to 1 MHz, e.g., in a range of about 10 kHz to about 500 kHz or in a range of about 20 kHz to about 100 kHz, can be especially advantageous in this regard. By way of example, the amplitude of the AC voltage applied to the reference electrode can be in a range of about 1 millivolt to about 3 volts, e.g., 0.5 volts to 1 volt. Further, in some cases, the voltage applied to the reference electrode can have an AC component and a DC offset, where the DC offset can be in a range of about −40 volts to about +40 volts, e.g., −1 volt to about +1 volt.

By way of illustration, FIG. 4C schematically depicts a combination of an AC voltage 3010 and a DC offset voltage 3012 applied to the reference electrode, resulting in voltage 3014. By way of example, the DC offset voltage can extend from about −10 V to about 10 V (e.g., from −1 V to about 1 V), and the applied AC voltage can have the frequencies and amplitudes disclosed above.

Without being limited to any particular theory, in some embodiments, it is expected that the application of such a voltage 3014 to the reference electrode 705 can minimize, and preferably eliminate, an effective capacitance associated with a sample, e.g., a liquid sample, with which the functionalized graphene layer is brought into contact as the sample is being tested, thereby facilitating the detection of a change in the resistance of the underlying graphene layer in response to the interaction of the antibodies 703 with a respective antigen. In some cases, the effective capacitance of the sample can be due to ions present in the sample and/or the formation of a liquid double layer over the functionalized graphene layer.

The sensors and the methods of the present teachings can be employed to detect the presence of hemoglobin protein in a variety of samples, such as urine, blood or feces. In some embodiments, the detection of the hemoglobin is indicated when the hemoglobin is present in a sample above a threshold that allows positive identification thereof based on the limit-of-detection (LOD) of the sensor. By way of example, in some embodiments, the threshold for the detection of hemoglobin can be about 10 μg·Hb/g·feces.

In some embodiments, a sensor according to the present teachings can include an array of sensing elements whose signals can be averaged to generate a resultant signal indicative of the presence or absence of hemoglobin protein (e.g., above a predefined threshold) in a sample, e.g., human feces.

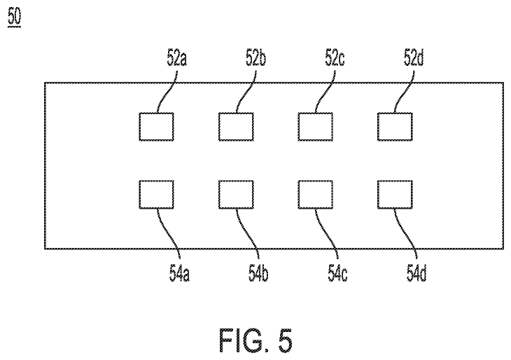

By way of example, FIG. 5 schematically depicts such a sensor 50 having a plurality of sensing elements 52a, 52b, 52c, and 52d (herein collectively referred to as sensing elements 52), and sensing elements 54a, 54b, 54c, and 54d (herein collectively referred to as sensing elements 54). Each of the sensing elements 52 and 54 includes a graphene layer functionalized with an anti-hemoglobin antibody and has a structure similar to that discussed above in connection with sensor 400. In some embodiments, the different sensing elements can be functionalized with different types of anti-hemoglobin antibodies. In some embodiments, the signals generated by the sensing elements 52 can be averaged to generate a resultant signal. Further, in some embodiments, at least one of the sensing elements 52 can be configured as a calibration sensing element to allow quantification of hemoglobin present in a sample. By way of example, the calibration can be achieved by utilizing a calibrated sample and detecting a change in at least one electrical property of the functionalized graphene layer in response to exposure to the calibration sample.

In use, a fecal sample can be mixed with a suitable liquid reagent and the mixture can be introduced into a sensor according to the present teachings. By way of example, the liquid reagent can be an aqueous buffer for dissolving a stool sample. By way of example, in some embodiments, a phosphate buffer (e.g., 1 g stool/25 ml of phosphate buffer) can be employed. The electrical resistance of the antibody-functionalized graphene layer can be monitored and analyzed to determine whether hemoglobin with a concentration above a predefined threshold is present in the fecal sample. For example, a change in the DC resistance of the antibody-functionalized graphene layer above a pre-defined threshold can be correlated with the detection of hemoglobin protein in the fecal sample.

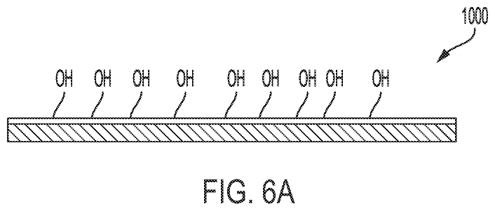

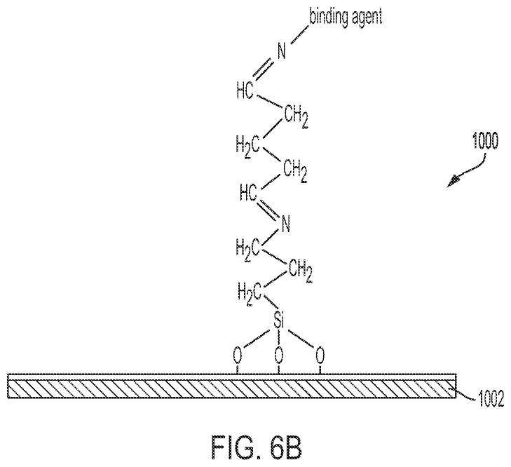

With reference to FIGS. 6A and 6B, in some embodiments, the sensor can include a hydroxyl-functionalized graphene layer 1000 that is further functionalized with anti-human hemoglobin antibodies via a molecule containing an aldehyde moiety.

More specifically, with reference to FIG. 6B, in this embodiment, the hydroxyl-functionalized graphene layer can be incubated with 2% 3-Aminopropyl triethoxysilane (APTES) in 95% ethanol for 1 hour to allow for aqueous silanization of the surface. The graphene layer can then be incubated in 2.5% glutaraldehyde in milli-Q water for a few hours (e.g., for 2 hours). This incubation can create aldehyde groups (—COH), which can react with amine groups (—NH2) of the antibody, e.g., via a covalent bond, thus coupling the antibody to the hydroxyl-functionalized graphene layer.

Similar to the previous embodiment, in this embodiment, the graphene layer can be initially deposited on an underlying substrate 1002. The underlying substrate 1002 can be, for example, a semiconductor, such as silicon, or a polymeric substrate, e.g., plastic.

Though not shown in FIG. 6B, similar to the above sensor 700, the sensor 1000 includes metallic pads that can allow application of an electrical signal (e.g., a current or a voltage) to the antibody-functionalized graphene layer and monitor at least one electrical property of the antibody-functionalized graphene layer, e.g., its DC electrical resistance.

Although colonoscopy remain the gold standard for early detection of colorectal cancer, the detection of hemoglobin in a fecal sample is a valuable screening tool that can be used alone or in combination with genetic screening for the detection of colorectal cancer and premalignant colorectal neoplasia.

In some embodiments, the high sensitivity of a graphene-based sensor according to the present teachings, e.g., characterized by an LOD of better than 10 μg·Hb/g·feces, for detecting hemoglobin protein in a stool sample can allow using only hemoglobin protein as a biomarker for the detection of colorectal cancer and pre-malignant colorectal neoplasia.

Another advantage of a graphene-based sensor according to the present teachings is that it can be utilized at the point of sample collection. As such, it obviates the need for mixing the sample with stabilizing reagents that can potentially interfere with the test results.

Further, in some cases, the detection of hemoglobin protein in a stool sample using a sensor according to the present teachings can be combined with genetic screening for the detection of colorectal cancer and pre-malignant colorectal neoplasia.

By way of example, such a genetic screening test can rely on detecting KRAS mutations, which have been detected in up to 35% of colorectal cancers and premalignant colorectal neoplasia.

Some known genetic screening modalities employ a multi-target approach for detecting DNA markers and hemoglobin can be used for detecting colorectal cancer and pre-malignant colorectal neoplasia. For example, in some known methods of genetic screening three independent categories of biomarkers are monitored. One such category of biomarkers includes epigenetic DNA changes in aberrant promoter regions of genes associated with DNA methylation. An example of methylated gene targets include N-Myc Downstream-Regulated Gene 4. Another category of biomarkers include DNA point mutations in the v-Ki-ras2 Kirsten rat sarcoma viral oncogene homolog (KRAS) gene, which encodes a small GTPase that is activated transiently as a response to (NDRG4) and the Bone Morphogenetic Protein 3 (BMP3). In one known approach, bisulfite conversion of non-methylated cytosine residues to uracil in the DNA sequence is employed to enable sensitive detection of hypermethylation associated with colorectal cancer and pre-malignant colorectal neoplasia.

Another biomarker for detection of colorectal cancer and premalignant colorectal neoplasia is a hemoglobin protein, which can be associated with chronic bleeding. The results from hemoglobin assays using a sensor according to the present teachings as well as abnormal methylation and mutation can collectively provide a positive or negative indication of colorectal cancer and pre-malignant colorectal neoplasia.

The following protocol is known for testing a DNA sample extracted from an individual's stool to determine the presence of biomarkers associated with colorectal cancer and pre-malignant colorectal neoplasia. In one approach, a stool sample is collected, and Quantitative Allele-specific Real-time Target and Signal Amplification (QuARTSTM) technology is employed for amplification and detection of methylated target DNA (NDRG4, BMP3), KRAS point mutations and ACTM (a reference gene that can be used for quantitative estimation of the total amount of human DNA in a sample). Multi-plexed QuARTS reactions can be processed using a real-time cycler with each biomarker (NDRG4, BMP3, KRAS, and ACTB) being monitored separately via independent fluorescent detection channels. Further details regarding extraction and detection of biomarkers associated with colorectal cancer can be found in U.S. Pat. No. 10,301,680, which is herein incorporated by reference in its entirety.

A sensor according to the present teachings can be used for detection of hemoglobin protein in the stool sample. Control samples can be used to ensure the reliability of the test results.

As noted above, in some embodiments, a sensor according to the present teachings can include a microfluidic channel for guiding a sample, e.g., a stool sample, from an inlet port, which receives a sample, to an outlet port through which the sample is delivered to a graphene-based sensing element according to the present teachings. In some such embodiments, such a microfluidic channel can include passive and/or active mixing elements for mixing the sample as the sample passes through the microfluidic channel.

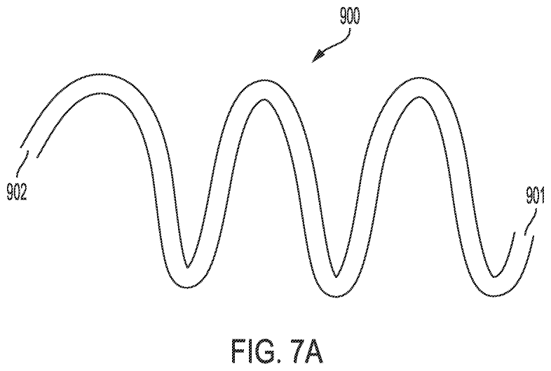

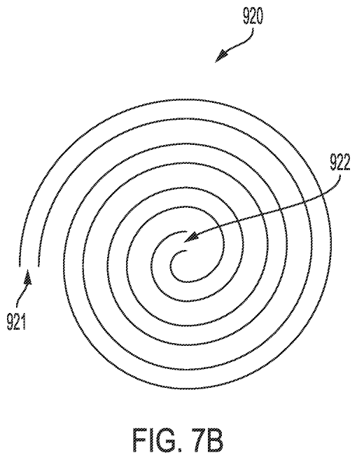

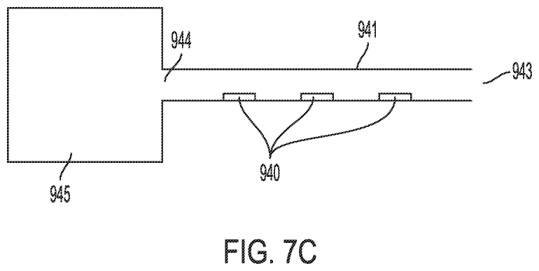

By way of example, FIG. 7A schematically depicts such a microfluidic channel 900 that has a serpentine shape extending from an inlet port 901 to an outlet port 902, where the serpentine shape of the microfluidic channel provides passive mixing of the sample as the sample passes through the channel. FIG. 7B shows another microfluidic channel 920 that has a spiral shape extending from an inlet port 921 to an outlet port 922, where the serpentine shape of the channel provides passive mixing of a sample passing through it. Other shapes, such as herringbone, can also be employed for a microfluidic channel that can provide mixing of a sample (e.g., a stool sample). Further, in some embodiments, obstacles can be provided in the microfluidic channel, instead of or in addition to configuring the shape of the channel, to provide mixing of a sample as it passes through the channel.

Yet, in other embodiments, a sensor according to the present teachings can include active mixing elements, micro-fluidic pumps, for mixing a sample, e.g., a stool sample. By way of example, as shown schematically in FIG. 7C, in some other embodiments, one or more piezo electric elements 940 can be disposed in the microfluidic channel 941, which extends from an inlet port 943 to an outlet port 944, and delivers a sample to graphene-based sensing element 945 according to the present teachings. The piezoelectric elements 940 can be actuated to cause mixing of a sample as it passes through the microfluidic channel 941.

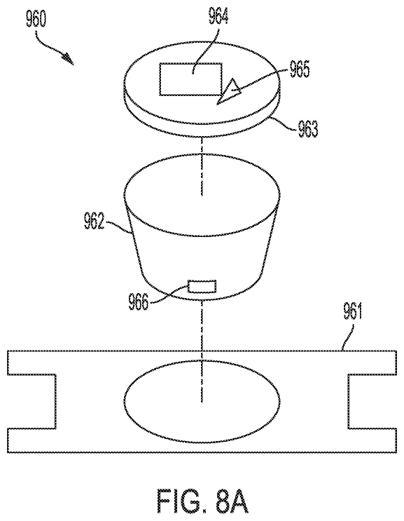

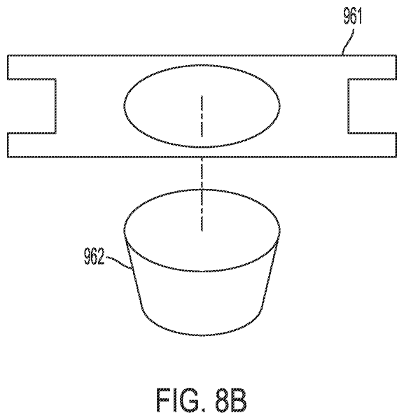

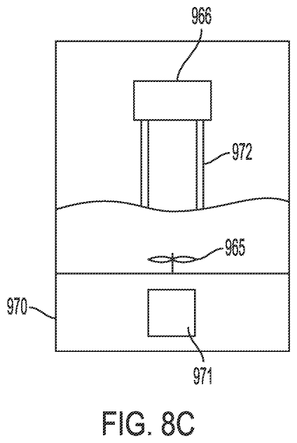

A variety of devices can be used to collect and process a sample, such as a stool sample. For example, with reference to FIGS. 8A, 8B, and 8C, a system 960 according to an embodiment of the present teachings for collecting a stool sample and testing the sample for presence of hemoglobin protein therein includes a toilet seat adapter 961 and a stool sample collection tube 962. The sample collection tube 962 can removably and replaceably engage with a toilet seat adapter 961 such that their respective openings are substantially aligned, thereby allowing the collection of a stool sample in the sample collection tube 962.

The system 960 further includes an auto-lock lid 963 that can be removably and replaceably coupled with the collection tube 962. In this embodiment, the lid 963 includes a pouch 964 in which one or more reagents can be stored for processing the stool sample as discussed in more detail below. Further, the lid 963 can include a mixing element 965, which is in the form a blade in this embodiment, which can be activated in a manner discussed herein to grind the sample. In this embodiment, in addition to the pouch 964 and the mixing element 965, a graphene-based sensing element according to the present teachings as well as the electronics for measuring an electrical property of the sensing element in response to exposure to a sample as well as an analysis module for analyzing the measured electrical property of the graphene-based sensing element are contained within the lid (herein collectively referred to as element 966). The graphene-based sensing element as well as the electronics for measuring the electrical property of the sensing element and the analysis module can be implemented in a manner discussed above. In other embodiments, the lid includes only the pouch containing one or more reagents for processing the sample as well as the mixing element while the sensing elements, the electronics and the analysis module are provided separately.

In use, a stool sample can be introduced into the collection tube 962 and the lid 963 can be attached to the collection tube 962. Subsequently, as shown in FIG. 8C, the collection tube 962 can then be inverted and coupled to a base 970 in which an actuator 971, for example, a motor, is disposed. The actuator 971 can actuate the mixing element 965 (i.e., the blade), which, upon actuation, punctures the pouch 964, thereby releasing the reagent(s) contained in the pouch 964 (e.g., a buffer) into the collection tube 962. The mixing of the stool sample with the reagent(s) as well as the mixing action of the mixing element processes the sample for introduction onto the sensing element 966.

With reference to FIG. 8C, in this embodiment, a plurality of wicking elements 972 can draw the processed sample into the sensing element 966.

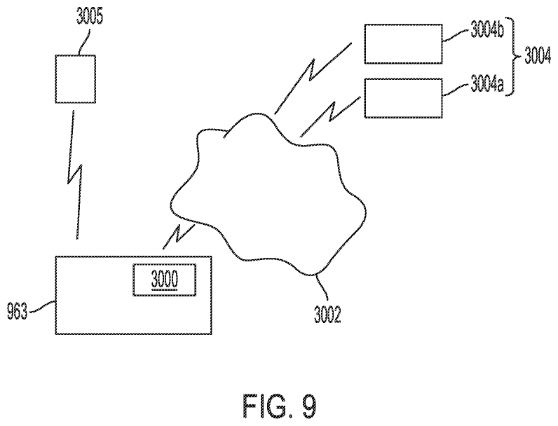

As shown in FIG. 9, in some embodiments, the electronics within the lid 963 can include a communication module 3000 (e.g., Wi-Fi) that can communicate the analysis results, via a network 3002 (e.g., the Internet) to one or more remote devices 3004, such as a physician's computer or mobile phone 3004a, and/or to an electronic health record (EHR) system 3004b. Further, in some embodiments, the communication module can allow communicating the results to a local device, e.g., a mobile phone 3005, using any suitable communications protocol, such as Bluetooth®.

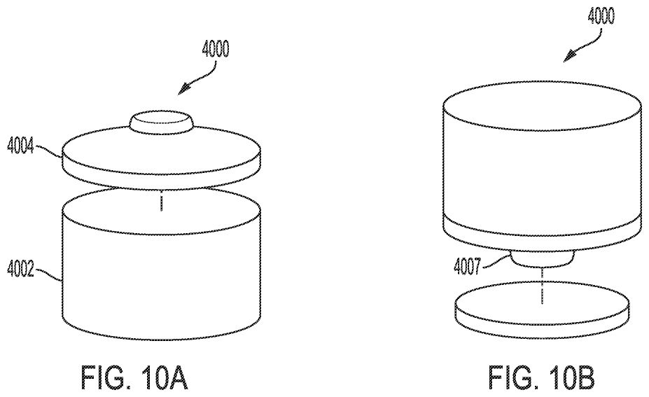

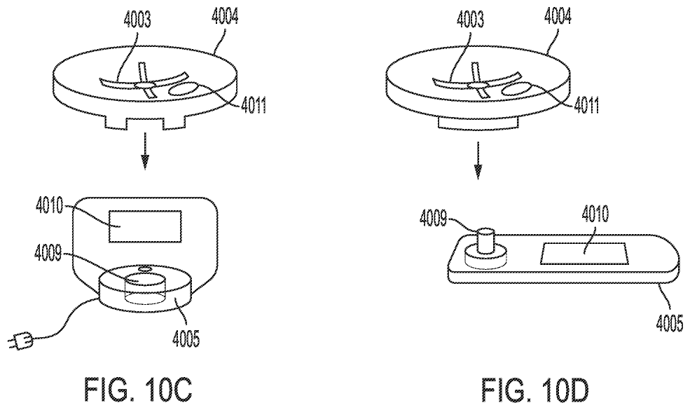

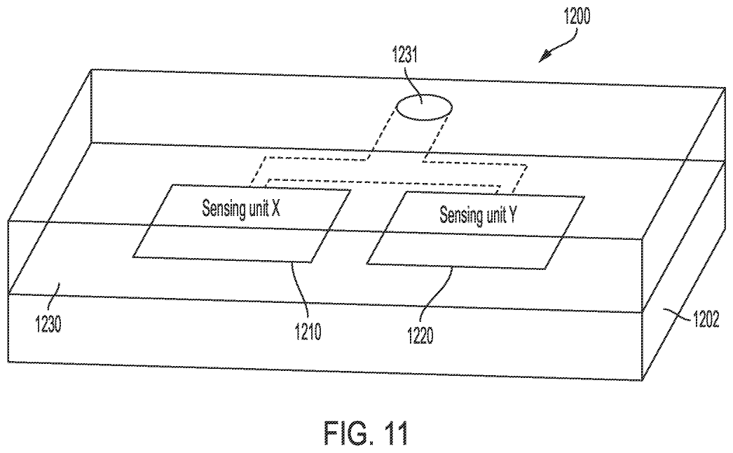

By way of further illustration, with reference to FIGS. 10A, 10B, 10C, and 10D, a system 4000 according to an embodiment includes a stool sample collection container 4002 and a lid 4004 that can removably and replaceably engage with the sample collection container to seal the container once a stool sample is placed in the container 4002.

In this embodiment, the lid 4004 includes a mixing element (e.g., an impeller or a magnet) 4003 that can be used to mix the sample once it is collected. A pouch 4011 is provided in the lid 4004 for storing one or more processing reagents for processing a sample. The pouch 4011 can be punctured by the mixing element 4003 once actuated to release its content into the container 4002 for mixing with a sample contained in the container 4002.

In this embodiment, the lid 4004 includes a connector 4007 provided on its external surface that can engage with a mating connector 4009 (e.g., a receptacle for receiving the connector 4007) provided in a base 4005 in which an actuator for activating the mixing element 4003 is disposed. The mating of the connectors allows application of energy (e.g., electrical energy) to the mixing element 4003 in order to activate the mixing element 4003. The activation of the mixing element 4003 causes mixing (e.g., grinding of the sample in some embodiments in which the mixing element 4003 is in the form of a blade). While the container 4002 and the associated lid 4004 are disposable items, the base 4005 can be made to be resusable.

In this embodiment, a display 4010 is provided that can present the results of a test to a user. Various components of the system, such as the container 4002, the lid 4004, and the display 4010 can be disposable (e.g., for home use) while the base 4005 can be reusable. In other embodiments, the base 4005 can also be disposable.

In other embodiments in which the sensing element is not incorporated in the lid, the lid can be removed and at least a portion of the processed sample can be extracted from the tube, e.g., via a syringe or other suitable device, and introduced into a sensor according to the present teachings.

In some embodiments, isothermal DNA amplification can be employed to amplify the DNA and hence facilitate genetic screening for biomarkers associated with colorectal cancer or pre-malignant neoplasia. A variety of approaches for performing isothermal amplification are known in the art. For example, some such approaches rely on using DNA polymerase to separate the strands of a double-stranded DNA (dsDNA) after initiation at a primer. A variety of other approaches are also known. For example, one such approach employs single stranded DNA binding protein gp32 from bacteriophage T4 and a strand-displacing DNA polymerase. Further details regarding this approach for isothermal amplification of DNA (as well as RNA) can be found in Scientific Reports (7: 8497, 2017), which is herein incorporated by reference in its entirety.

A sensor according to the present teachings provides a fast, cost-effective and easy-to-use tool that can be employed to detect hemoglobin in a biological sample, e.g., a fecal sample.

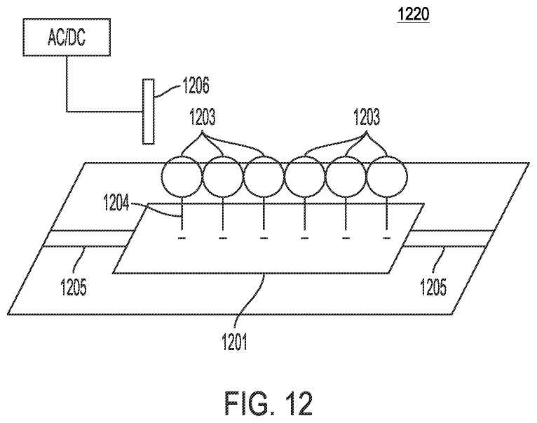

With reference to FIGS. 11 and 12, in some embodiments, a sensor 1200 according to the present teachings can include at least two sensing units 1210 and 1220, wherein the sensing unit 1210 is configured as a graphene-based sensor according to the present teachings to detect the presence of hemoglobin in a sample, such as a fecal sample. The sensor unit 1220 is a graphene-based sensor that is configured to detect one or more mutations (e.g., deletions or additions) in the generic material(s) present in the sample under investigation. As discussed in more detail below, in some embodiments, a portion of a sample, e.g., a fecal sample, processed in a manner disclosed herein can be introduced into the sensing unit 1210 and another portion of the sample can be introduced into the sensing unit 1220. As discussed above, the presence of hemoglobin beyond a certain threshold together with identification of one or more mutations in the genome can indicate the detection of colorectal cancer or pre-malignant colorectal neoplasia.

With reference to FIG. 12, similar to the graphene-based sensing units discussed above, the graphene-based sensing unit 1220 includes a graphene layer 1201 that is disposed over an SiO₂ layer, which is in turn disposed on an underlying silicon layer 1202. In this embodiment, the graphene layer 1201 is functionalized with a catalytically deactivated Cas9 (dCas9) CRISPR complex 1203 (e.g., Cas9 (dCas9) CRISPR complex). As discussed below, a target DNA sequence (e.g., a target DNA sequence containing a mutation of interest) can selectively hybridize to the CRISPR complex and such hybridization of the target DNA sequence (a portion of one strand of the sample DNA) modulates at least one electrical property of the functionalized graphene layer 1201, which can indicate the presence of a mutation (e.g., a mutation associated with colorectal neoplasia).

In this embodiment, the graphene layer 1201 can be functionalized with a molecular linker 1204 (e.g., 1-pyrenebutanic acid) via π-π aromatic stacking with the underlying graphene layer 1201, e.g., in a manner discussed above. The dCas9 complex 1203 can then be anchored to the molecular linker 1204, e.g., via carbodiimide cross-linking chemistry. The portions of the graphene layer 1201 that is not functionalized can then be passivated, e.g., by blocking those portions with blocking agents such as those discussed above (e.g., amino-PEGS-alcohol and/or ethanolamine hydrochloride) to prevent non-specific binding of non-target molecules to the unfunctionalized portions of the graphene layer 1201. The dCas9 complex 1203 can then be coupled to a single strand guide RNA (sgRNA) that include a nucleotide sequence that is complementary to the target DNA sequence of interest.

A plurality of conductive electrodes 1205 is deposited on the graphene layer to allow the measurement of at least one electrical property of the functionalized graphene layer 1201 (e.g., its impedance) in response to interaction of a sample with the functionalized graphene layer 1201. In some such embodiments, one electrode can function as a source electrode and the opposed electrode can function as a drain electrode.

Further, similar to the previous embodiments, the sensor 1200 can include a gate (reference) electrode 1206 to which a DC voltage or a AC voltage in combination with a DC ramp voltage can be applied (e.g., in a manner discussed above (See, e.g., FIGS. 4A and 4B)).

For example, as shown in FIG. 12, a current source can apply a predefined constant current to the graphene layer 1201 while an AC/DC voltage source applies an AC voltage together with a DC ramp voltage to the gate electrode 1206. Similar to the above embodiment, the sensor 1200 includes a fluidic manifold 1230 that allows receiving, via an inlet 1231, thereof, a sample under investigation. The fluidic manifold 1230 includes a plurality of internal channels that allow the distribution of a portion of the received sample to the sensing unit 1210 and another portion of the received sample to the sensing unit 1220.

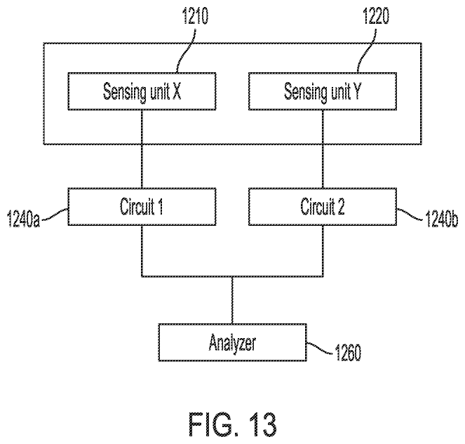

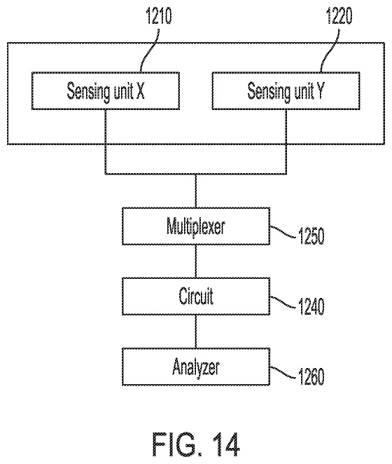

In the embodiment shown in FIG. 13, two measurement circuits 1240a and 1240b allow monitoring the voltage across the functionalized graphene layers of the sensing units 1210 and 1220. As shown in FIG. 14, in some embodiments, rather than providing a dedicated measurement circuit for each sensing unit, a single measurement circuit 1240 is provided, which is coupled via a multiplexer 1250, to the sensing units for monitoring the voltages across the sensing units 1210/1220 in different time intervals (e.g., during successive time intervals).

Referring again to FIGS. 13 and 14, an analyzer 1260 in communication with the measurement circuitry 1240 can receive the signals generated by the sensing units 1210/1220 (e.g., voltage measurements across the functionalized graphene layers) and process those signals to determine whether hemoglobin above the limit-of-detection (LOD) of the sensing unit 1210 is present in the sample under investigation and whether the signal generated by the sensing unit 1220 is indicative of the presence of a target mutation in the DNA present in the sample.

In some embodiments, prior to the introduction of a sample into the sensor, DNA amplification techniques, such as those discussed above, can be employed, e.g., in a manner discussed above, to amplify the DNA within the sample.

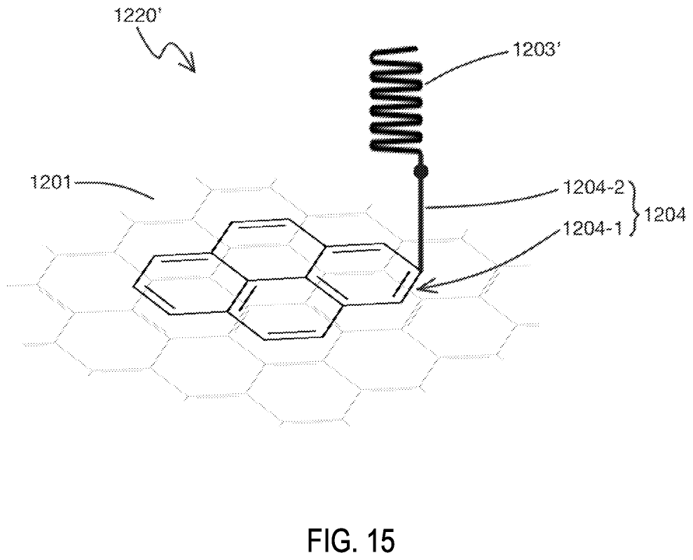

FIG. 15 schematically depicts another embodiment of the sensing unit that can be included in the sensor 1200 along with the hemoglobin sensing unit 1210 for detecting hemoglobin in a sample, e.g., a fecal sample, such as those disclosed herein as well as a sensing unit 1220 that is configured to detect mutations, including single-base mutations, in the DNA and/or RNA present in the sample. Unlike the previous embodiment, in this embodiment of the sensing unit 1220′, an oligonucleotide-binding element other than a CRISPR complex can be coupled to the graphene layer 1201 as an oligonucleotide probe (e.g., a DNA or RNA probe) element for detecting a target oligonucleotide (e.g., DNA or RNA) segment (e.g., a portion of a single strand of a DNA sequence). For example, in this embodiment, the graphene layer 1201 can be functionalized with a plurality of oligonucleotides 1203′ having a nucleotide sequence that is complementary to that of a target nucleotide sequence, e.g., a DNA or an RNA sequence having a mutation, e.g., a single base mismatch with the nucleotides of the probe oligonucleotide.

In this embodiment, the oligonucleotides 1203′ can be coupled to the graphene layer 1201 using a linker 1204, such as 1-pyrenebutonic acid succinimidyl discussed above. Such a linker can be coupled to the graphene layer 1201 via π stacking of its pyrene group 1204-1 onto the graphene surface while the succinimide portion 1204-2 of the linker 1204 can covalently bind to an amine moiety of the oligonucleotide 1203′. In use, the hybridization of a target oligonucleotide sequence (e.g., a target single strand DNA segment) to the probe oligonucleotide 1203′ coupled to the graphene layer 1201 can cause a change in at least one electrical property of the graphene layer 1201 (e.g., its electrical impedance, such as its DC resistance), which can be detected in a manner disclosed herein.

In other embodiments, other types of oligonucleotide binding elements can be employed. Some examples of such oligonucleotide binding elements include, without limitation, zinc fingers, and the DNA binding domains of TALENs (transcription activator-like effector nucleases), among others.

### EXAMPLE

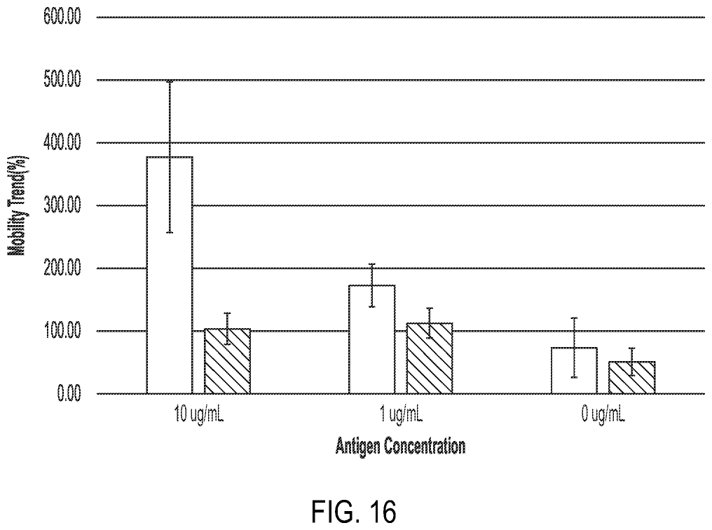

A prototype sensor according to the present teachings was fabricated as described above and tested using samples having various concentrations of hemoglobin antigens. The graphene layer of the sensor was functionalized with mouse monoclonal anti-hemoglobin antibodies (7E1F) marketed by Abcam under the product code Ab77125. Further, the unfunctionalized portions of the graphene layer were passivated using polyethylene glycol (PEG) and ethanolamine. A control sensor was also fabricated in which the graphene layer was functionalized with an isotype control antibodies. Both sensors were loaded with samples including hemoglobin antigens in concentrations of 0 μg/mL, 1 μg/mL, and 10 μg/mL, and the electron mobility was measured. As shown in Table 1 and FIG. 16, the prototype sensor exhibited a change in electron mobility (i.e., a percentage change in conductivity or electron mobility between a buffer containing no antigen and a sample containing hemoglobin antigens) in response to interaction with hemoglobin that was statistically higher than that exhibited by the control sensor.

TABLE 1
Electron mobilities for different hemoglobin (antigen) concentrations

| | 10 μg/mL | | 1 μg/mL | | 0 μg/mL | |
|---|---|---|---|---|---|---|
| | Average | St Dev. | Average | St Dev. | Average | St Dev. |
| Hemoglobin | 377.25 | 120.09 | 172.89 | 34.14 | 73.39 | 47.17 |
| mAb | | | | | | |
| Isotype control | 103.68 | 24.71 | 112.78 | 23.87 | 51.11 | 21.99 |

Those having ordinary skill in the art will appreciate that various changes can be made to the above embodiments without departing from the scope of the present invention.

### Conclusion and General Terminology

In various embodiments, one or more of disclosed modules are implemented via one or more computer programs for performing the functionality of the corresponding modules, or via computer processors executing those programs. In some embodiments, one or more of the disclosed modules are implemented via one or more hardware modules executing firmware for performing the functionality of the corresponding modules. In various embodiments, one or more of the disclosed modules include storage media for storing data used by the module, or software or firmware programs executed by the module. In various embodiments, one or more of the disclosed modules or disclosed storage media are internal or external to the disclosed systems. In some embodiments, one or more of the disclosed modules or storage media are implemented via a computing “cloud”, to which the disclosed system connects via a network connection and accordingly uses the external module or storage medium. In some embodiments, the disclosed storage media for storing information include non-transitory computer-readable media, such as a CD-ROM, a computer storage, e.g., a hard disk, or a flash memory. Further, in various embodiments, one or more of the storage media are non-transitory computer-readable media that store data or computer programs executed by various modules, or implement various techniques or flow charts disclosed herein.

The above detailed description refers to the accompanying drawings. The same or similar reference numbers may have been used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

The foregoing description of the embodiments has been presented for purposes of illustration only. It is not exhaustive and does not limit the embodiments to the precise form disclosed. While several exemplary embodiments and features are described, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. As used herein, the singular forms “a,” “an,” and “the” may include the plural forms unless the context clearly dictates otherwise. Further, the term “coupled” does not exclude the presence of intermediate elements between the coupled items. Also, stating that a feature may exist indicates that the feature may exist in one or more embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or. Moreover, if these terms are used, a subset of a set may include one or more than one, including all, members of the set.

The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments, and may also include other parts not described in the embodiments. Accordingly, the embodiments are not limited to the above-described details, but instead are defined by the appended claims in light of their full scope of equivalents. Further, the present disclosure is directed toward all novel and non-obvious features and aspects of the various disclosed embodiments, alone and in various combinations and sub-combinations with one another.

While the present disclosure has been particularly described in conjunction with specific embodiments, many alternatives, modifications, and variations will be apparent in light of the foregoing description. It is therefore contemplated that the appended claims will embrace any such alternatives, modifications, and variations as falling within the true spirit and scope of the present disclosure.

What is claimed is:

1. A sensor for detecting hemoglobin in a sample, comprising:
a graphene layer;
a plurality of binding agents coupled to said graphene layer to generate a functionalized graphene layer, wherein said binding agents bind to hemoglobin protein; and
a plurality of electrical conductors electrically coupled to said functionalized graphene layer for measuring at least one electrical property of said functionalized graphene layer.

2. The sensor of claim 1, wherein said binding agents comprise antibodies exhibiting specific binding to said hemoglobin protein.

3. The sensor of claim 1, wherein said binding agents comprise aptamers exhibiting specific binding to said hemoglobin protein.

4. The sensor of claim 1, further comprising a reference electrode for applying a reference AC signal to said functionalized graphene layer.

5. The sensor of claim 4, wherein said reference AC signal has a frequency 1 kHz to 1 MHz.

6. The sensor of claim 1, wherein said sample comprises a biological sample.

7. The sensor of claim 6, wherein said biological sample comprises human feces.

8. The sensor of claim 7, wherein said sensor has a detection limit of 10 μg·Hb/g·feces for detecting hemoglobin in human feces.

9. The sensor of claim 1, wherein said binding agents comprise an anti-human hemoglobin antibody.

10. The sensor of claim 1, wherein said binding agents are coupled to the graphene layer via a plurality of linkers.

11. The sensor of claim 10, wherein each of said linkers is covalently attached at one end thereof to the graphene layer and at another end to at least one hemoglobin protein.

12. The sensor of claim 10, wherein said linkers comprise 1-pyrenebutonic acid succinimidyl ester.

13. The sensor of claim 1, wherein said graphene layer is functionalized with a plurality of hydroxyl groups.

14. The sensor of claim 13, wherein said binding agents are coupled to one or more of said plurality of hydroxyl groups via a plurality of aldehyde moieties.

15. A method of detecting hemoglobin in a biological sample, comprising:
applying the biological sample to a graphene layer functionalized with a plurality of binding agents that bind to hemoglobin;
measuring at least one electrical property of the functionalized graphene layer; and
using said measured electrical property to determine whether hemoglobin is present in said sample.

16. The method of claim 15, wherein said biological sample comprises human feces.

17. The method of claim 15, wherein said biological sample comprises urine.

18. The method of claim 15, wherein said at least one electrical property of the functionalized graphene layer comprises a DC electrical resistance thereof.

19. The method of claim 15, wherein said step of using the measured electrical property comprises monitoring a change in said electrical property in response to interaction of said sample with the functionalized graphene layer.

20. The method of claim 15, further comprising utilizing genetic screening in combination with detection of said hemoglobin in said biological sample for detection of colorectal cancer.

21. A method of fabricating a sensor for detecting hemoglobin protein in a biological sample, comprising:
covalently attaching a plurality of linkers to a graphene layer deposited on an underlying substrate; and
covalently coupling a plurality of binding agents exhibiting specific binding to hemoglobin protein to said linkers.

22. A disposable cartridge for detecting hemoglobin protein in a sample, comprising:
a microfluidic component having an inlet port for receiving a sample and an exit port; and
a sensor fluidically coupled to said microfluidic component to receive at least a portion of said sample from said exit port,
wherein said sensor comprises:
a graphene layer;
a plurality of binding agents coupled to said graphene layer to generate a functionalized graphene layer, wherein said binding agents bind to hemoglobin protein; and
a plurality of electrical conductors electrically coupled to said functionalized graphene layer for measuring at least one electrical property of said functionalized graphene layer.

23. The disposable cartridge of claim 22, wherein said microfluidic component comprises a polymeric material.

24. The disposable cartridge of claim 23, wherein said polymeric material comprises any of PDMS and PMMA.

25. A method of screening for any of colorectal cancer and pre-malignant colorectal neoplasia, comprising:
collecting a stool sample from a subject;
dissolving the collected stool sample in a buffer solution to form a testing sample;
introducing said testing sample onto a sensing element comprising a graphene layer functionalized with a plurality of binding agents exhibiting specific binding to hemoglobin protein;
measuring at least one electrical property of said functionalized graphene layer in response to exposure thereof to said testing sample; and
determine whether hemoglobin above a threshold associated with a sensitivity of said sensing element is present in said testing sample.

26. The method of claim 25, further comprising quantifying concentration of the hemoglobin in said testing sample.

27. The method of claim 25, correlating said quantified concentration with presence or absence of colorectal cancer or pre-malignant colorectal neoplasia.

28. The method of claim 25, wherein said testing sample lacks a stabilizing reagent.

29. The method of claim 25, further comprising utilizing at least one genetic screening method in combination with detection of said hemoglobin protein.

30. The method of claim 29, wherein said genetic screening method comprises detecting one or more KRAS mutations.

31. A system for testing a stool sample, comprising:
a collection tube for receiving a stool sample through an opening thereof;
a lid for removably and replaceably coupling to the said opening of the collection tube, said lid having a pouch for storing one or more reagents for processing said sample and a mixing element, which upon actuation, punctures the pouch to release said one or more reagents; and
a sensor for detecting hemoglobin protein in said sample, wherein said sensor comprises:
a graphene layer;
a plurality of binding agents coupled to said graphene layer to generate a functionalized graphene layer, wherein said binding agents bind to hemoglobin protein; and
a plurality of electrical conductors electrically coupled to said functionalized graphene layer for measuring at least one electrical property of said functionalized graphene layer.
